(19) United States
(12) Patent Application Publication
(10) Pub. No.: US 2009/0131781 A1
(43) Pub. Date: May 21, 2009

(54) DETERMINATION OF SUSCEPTIBILITY-INDUCED MAGNETIC FIELD GRADIENTS BY MAGNETIC RESONANCE

(75) Inventors: Hannes Dahnke, Hamburg (DE); Tobias Schaeffter, Blackheath (GB)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N. V., Eindhoven (NL)

(21) Appl. No.: 12/297,533

(22) PCT Filed: Apr. 2, 2007
(86) PCT No.: PCT/IB2007/051176

§ 371 Date: Oct. 17, 2008

(30) Foreign Application Priority Data
Apr. 21, 2006 (EP) 06112868.2
Jan. 18, 2007 (EP) 07100712.4

(51) Int. Cl. A61B 5/055

(52) U.S. Cl. 600/410

(57) ABSTRACT

The invention relates to a device for magnetic resonance imaging of a body (7). The device (1) comprises means (2) for establishing a substantially homogeneous main magnetic field in the examination volume, means (3, 4, 5) for generating switched magnetic field gradients superimposed upon the main magnetic field, means (6) for radiating RF pulses towards the body (7), control means (12) for controlling the generation of the magnetic field gradients and the RF pulses, means (10) for receiving and sampling magnetic resonance signals, and reconstruction means (14) for forming MR images from the signal samples. In accordance with the invention, the device is arranged to a) generate a series of MR echo signals (20) by subjecting at least a portion of the body (7) to an MR imaging sequence of RF pulses and switched magnetic field gradients, b) acquire the MR echo signals for reconstructing an MR image data set (21) therefrom, c) calculate a gradient map (22) by computing echo shift parameters (SPₓ, SP_{y}, SP_{z}) from subsets of the MR image data set, the echo shift parameters (SPₓ, SP_{y}, SP_{z}) indicating magnetic field gradient induced shifts of the echo positions in k-space, wherein each subset comprises a number (n) of spatially adjacent pixel or voxel values of the MR image data set (21).

### TECHNICAL FIELD

The invention relates to a device for magnetic resonance imaging of a body placed in an examination volume.

Furthermore, the invention relates to a method for MR imaging and to a computer program for an MR device.

### BACKGROUND AND RELATED ART

In magnetic resonance imaging (MRI) pulse sequences of RF pulses and switched magnetic field gradients are applied to an object (a patient) placed in a homogeneous magnetic field within an examination volume of an MR device. In this way, phase encoded magnetic resonance signals are generated, which are scanned by means of RF receiving antennas in order to obtain information from the object and to reconstruct images thereof. Since its initial development, the number of clinically relevant fields of application of MRI has grown enormously. MRI can be applied to almost every part of the body, and it can be used to obtain information about a number of important functions of the human body. The pulse sequence, which is applied during an MRI scan, plays a significant role in the determination of the characteristics of the reconstructed image, such as location and orientation in the object, dimensions, resolution, signal-to-noise ratio, contrast, sensitivity for movements, etcetera. An operator of an MRI device has to choose the appropriate sequence and has to adjust and optimize its parameters for the respective application.

An object having a magnetic susceptibility that deviates from the surrounding creates local inhomogeneities of the main magnetic field B₀. This applies to metallic objects (such as surgical instruments, implants or other devices), iron-containing substances like deoxygenated blood, or iron oxide based contrast agents or labeled cells. This also applies to empty regions within the imaged body since there is a strong susceptibility difference between the empty region (filled with gas) and the surrounding tissue. The exploitation of this effect is an important tool for different MR imaging applications ranging from contrast agent (e.g. SPIO) detection to the localization of devices (catheters, implantable stents, etc.).

Susceptibility contrast enhanced MR imaging is usually performed via T₂ or T₂* weighted sequences. With these sequences the contrast is created by signal losses at the site of a local magnetic field disturbance. In the images generated by these known techniques, dark image features that are due to field inhomogeneities can not be distinguished from features that are due to other effects leading to signal losses.

Several concepts of converting the dark image contrast into a positive (bright) contrast have been proposed. For example, EP 1 471 362 A1 discloses an MR method that is based on a gradient echo (GE) imaging sequence. In accordance with this known technique a certain imbalance of switched magnetic field gradients or additional gradients are applied in order to generate an MR image showing positive (bright) contrast between background tissue and objects producing local magnetic field inhomogeneities. A drawback of this known technique is that in order to obtain optimal positive image contrast, either prior knowledge about the strength of the susceptibility gradients is required, or at least an elaborate and time-consuming optimization procedure has to be performed.

Therefore, it is readily appreciated that there is a need for an improved device for magnetic resonance imaging for the generation of images with positive (bright) susceptibility contrast. It is consequently an object of the invention to provide an MR device that enables susceptibility imaging without prior optimization for obtaining the optimal positive contrast. A further object of the invention is to provide an MR device, which is able to produce images with positive susceptibility contrast without the use of special or unconventional MR imaging sequences.

### SUMMARY OF THE INVENTION

In accordance with the present invention, an MR device for magnetic resonance imaging of a body placed in an examination volume is disclosed, which comprises means for establishing a substantially homogeneous main magnetic field in the examination volume, means for generating switched magnetic field gradients superimposed upon the main magnetic field, means for radiating RF pulses towards the body, control means for controlling the generation of the magnetic field gradients and the RF pulses, means for receiving and sampling magnetic resonance signals, and reconstruction means for forming MR images from the signal samples. According to the invention, the device is arranged to

a) generate a series of MR echo signals by subjecting at least a portion of the body to an MR imaging sequence of RF pulses and switched magnetic field gradients,

b) acquire the MR echo signals for reconstructing an MR image data set therefrom,

c) calculate a gradient map by computing echo shift parameters from subsets of the MR image data set, the echo shift parameters indicating magnetic field gradient induced shifts of the echo positions in k-space, wherein each subset comprises a number of spatially adjacent pixel or voxel values of the MR image data set.

The MR device of the invention is arranged to acquire an MR image data set in steps a) and b) by means of a standard imaging sequence that is conventionally used for imaging of the anatomy of the examined body. The acquired MR image data set thus contains the complete anatomical information. In addition, a gradient map is calculated in step c) from the anatomical image data set. The gradient map contains quantitative information about the local susceptibility induced magnetic field gradient strength. This information can be used, for example, to generate a corresponding positive contrast image or to localize a metallic object within the examination volume without any additional measurement.

The basic idea of the invention is to use the information with regard to local field inhomogeneity that is contained in each subset of spatially adjacent pixels or voxels of the reconstructed MR image data set. The invention is based upon the insight that local (susceptibility induced) gradients act in addition to the switched magnetic field gradients during imaging, the local gradients causing shifts of the echo signal maxima in k-space. In accordance with the invention, a local echo shift parameter is calculated from a corresponding subset of pixels or voxels. This echo shift parameter is indicative of a shift of the echo position in k-space, wherein this shift stems from the magnetic field gradients affecting the pixels or voxels of the respective subset. Thus, the local gradient strength can be concluded from the echo shift parameter.

The susceptibility gradient map can be converted into a positive contrast image simply by assigning grey values to the echo shift parameters.

The device of the invention enables the derivation of the local magnetic field gradient distribution within the examination volume and the production of a positive susceptibility contrast image by mere post-processing of a conventional (2D or 3D) anatomical MR image data set. An optimal positive contrast imaging is achieved without the use of dedicated sequences and without additional optimization procedures.

Preferably, the device is further arranged in accordance with the invention to calculate the gradient map by computing Fourier transformations over the adjacent pixel or voxel values of each subset in step c). The echo shift parameters can then be computed by determining the positions of the maxima of the Fourier components for each subset. The positions of the maxima of the Fourier components correspond to the respective echo positions in k-space. Independent one-dimensional Fourier transformations may be computed over the adjacent pixel or voxel values in each spatial direction of the MR image data set. On this basis, the gradient map can be calculated by computing the strength and direction of the gradient from the echo shift parameters in the different spatial directions. In this way, the local gradient vectors are calculated. This allows for the analysis of the direction and of the distribution of anisotropy of the local magnetic field gradients.

In a practical embodiment of the invention, the gradient map may be calculated at a reduced spatial resolution as compared to the spatial resolution of the MR image data set. For example, if the echo shift parameters are calculated from subsets of n adjacent pixels or voxels, the spatial resolution of the susceptibility gradient map may be calculated at an n-fold lower resolution than the MR image data set.

The MR device of the invention may be arranged to compute echo shift parameters at first from subsets comprising a smaller number of spatially adjacent pixel or voxel values of the MR image data set. Then the echo shift parameters are computed again from subsets comprising a larger number of spatially adjacent pixel or voxel values. The accuracy of the calculation of the gradient map can be incrementally improved by this hierarchic approach.

It is a well-known fact that it is very important in MR imaging to establish a homogeneous main magnetic field B₀ within the examination volume in order to be able to acquire accurate, undistorted images of the examined portion of the patient's body. A common way to provide a homogeneous main magnetic field is to generate a static magnetic field B₀ by means of a main magnet and to generate an adjustable auxiliary magnetic field to compensate for inhomogeneities of the static magnetic field. The auxiliary magnetic field is generated by so-called shim coils whose shapes and current paths enable an effective compensation of inhomogeneities of the field generated by the main magnet. The process of correcting the static magnetic field B₀ by passing the appropriate shim currents through the shim coils is usually referred to as shimming. The shim current values determining the shim currents passed through each shim coil are usually determined once during a preparation phase. Consequently, local magnetic field gradients induced, e.g., by dynamically changing susceptibility effects (patient motion) can not be compensated for by conventional shimming strategies.

It is thus a further object of the invention is to provide an MR device which enables improved shimming of the main magnetic field distribution.

It is an insight of the invention that the gradient map obtained by the technique described herein before can advantageously be used to determine optimal shim current values for a region of interest. Thus, in accordance with the invention, shim current values are derived from the gradient map and corresponding shim currents are passed through the shim coils of the MR device for producing an auxiliary magnetic field to optimze the homogeneity of the main magnetic field within the examination volume. A user of the MR apparatus may interactively select a region of interest in which the shim of the main magnetic field is automatically determined from the acquired MR echo signals, i.e. no extra measurement is required. Shim current values for different regions can easily be determined from one and the same MR signal data set. This automatic shimming technique can advantageously be integrated in dynamic MR imaging methods and also real-time MR imaging methods in order to enable continuously updating the shim of the main magnetic field. Image distortions due to field imperfections are effectively minimized in this way, i.e. image quality is significantly improved.

In conventional MR systems, three-dimensional series polynomials, such as, e.g., Legendre polynomials, are used to model the auxiliary magnetic field generated by the shim coils, wherein each shim current value corresponds to one coefficient of the polynomial. It is an advantage of the invention that the linear shim current values can be derived directly from the gradient values within the region of interest and that a conventional set of shim coils can be used. For shimming within a given region of interest the shim current values may be determined, e.g., by averaging over the gradient values within this region.

The invention not only relates to a device but also to a method for magnetic resonance imaging of at least a portion of a body placed in an examination volume of an MR device. The method comprises the following steps:

a) generating a series of MR echo signals by subjecting at least a portion of the body to an MR imaging sequence of RF pulses and switched magnetic field gradients,

b) acquiring the MR echo signals for reconstructing an MR image data set therefrom,

c) calculating a gradient map by computing echo shift parameters from subsets of the MR image data set, the echo shift parameters indicating susceptibility induced shifts of the echo positions in k-space, wherein each subset comprises a number of spatially adjacent pixel or voxel values of the MR image data set.

A computer program adapted for carrying out the imaging procedure of the invention can advantageously be implemented on any common computer hardware, which is presently in clinical use for the control of magnetic resonance scanners. The computer program can be provided on suitable data carriers, such as CD-ROM or diskette. Alternatively, it can also be downloaded by a user from an Internet server.

### BRIEF DESCRIPTION OF THE DRAWINGS

The enclosed drawings disclose preferred embodiments of the present invention. It should be understood, however, that the drawings are designed for the purpose of illustration only and not as a definition of the limits of the invention. In the drawings

FIG. 1 shows an MR scanner according to the invention;

FIG. 2 shows a diagram illustrating the method of the invention.

### DETAILED DESCRIPTION

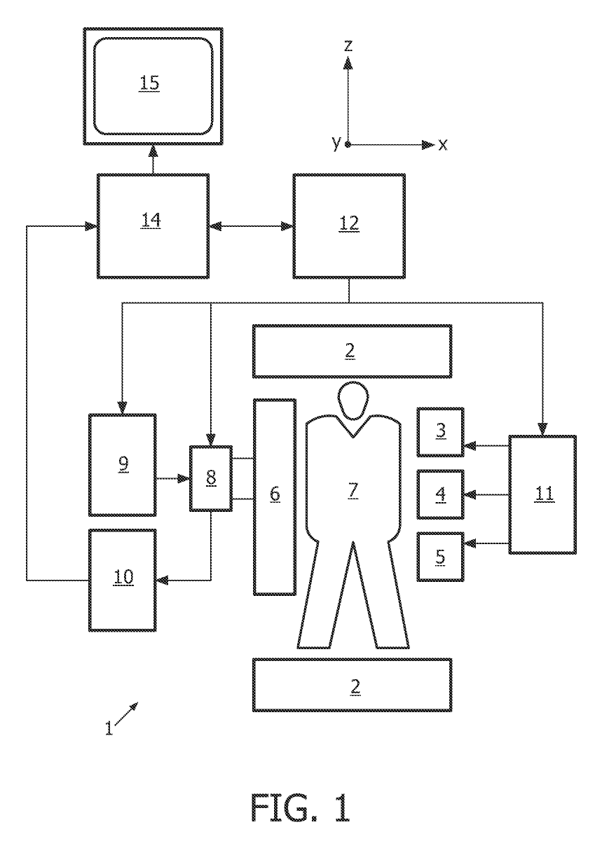

In FIG. 1 an MR imaging device 1 in accordance with the present invention is shown as a block diagram. The apparatus 1 comprises a set of main magnetic coils 2 for generating a stationary and substantially homogeneous main magnetic field and three sets of gradient coils 3, 4 and 5 for superimposing additional magnetic fields with controllable strength and having a gradient in a selected direction. Conventionally, the direction of the main magnetic field is labelled the z-direction, the two directions perpendicular thereto the x- and y-directions. The gradient coils 3, 4 and 5 are energized via a power supply 11. The imaging device 1 further comprises an RF transmit antenna 6 for emitting radio frequency (RF) pulses to a body 7. The antenna 6 is coupled to a modulator 9 for generating and modulating the RF pulses. Also provided is a receiver for receiving the MR signals, the receiver can be identical to the transmit antenna 6 or be separate. If the transmit antenna 6 and receiver are physically the same antenna as shown in FIG. 1, a send-receive switch 8 is arranged to separate the received signals from the pulses to be emitted. The received MR signals are input to a demodulator 10. The send-receive switch 8, the modulator 9, and the power supply 11 for the gradient coils 3, 4 and 5 are controlled by a control system 12. Control system 12 controls the phases and amplitudes of the RF signals fed to the antenna 6. The control system 12 is usually a microcomputer with a memory and a program control. The demodulator 10 is coupled to reconstruction means 14, e.g. a computer, for transformation of the received signals into images that can be made visible, e.g., on a visual display unit 15. Furthermore, the MR imaging device 1 comprises a set of three shim coils 16, 17, and 18. An auxiliary magnetic field is generated by shim currents passed through the shim coils 16, 17, and 18 via separate shim channels from a shim current supply 19. The strength of the shim currents is controlled by control system 12 to optimize the homogeneity of the main magnetic field. For the practical implementation of the invention, the MR device 1 comprises a programming for carrying out the above-described method.

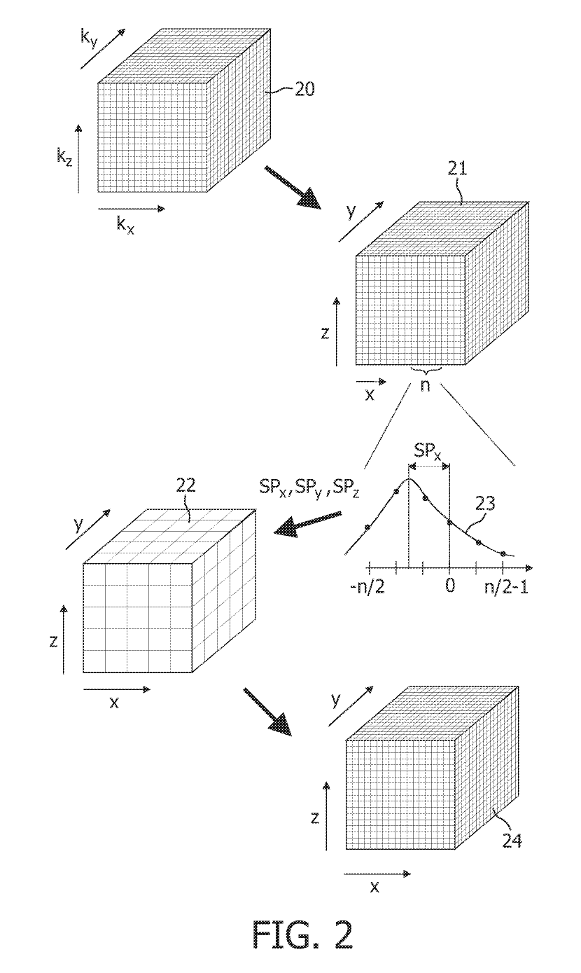

FIG. 2 illustrates the method of the invention as a diagram. In a first step, a 3D MR data set 20 is acquired by means of a conventional 3D gradient echo imaging sequence (for example 3D EPI). Then, the echo signal data set 20 is transformed into a (complex) 3D MR image data set 21 via standard image reconstruction techniques. As a next step, a three-dimensional gradient map 22 is calculated. For this purpose, 1D Fourier transformations are performed for subsets of n adjacent voxels separately in all three dimensions x, y, and z. In FIG. 2, the determination of a single gradient value in one spatial dimension is exemplarily shown. The 1D Fourier transform 23 comprises −n/2 to n/2−1 Fourier components. As can be seen in FIG. 2, the maximum of these Fourier components is shifted proportionally to the local magnetic field gradient acting in the direction of the Fourier transformation. From the discrete Fourier components 23, the position of the maximum is determined at sub Fourier component resolution by means of a least squares fitting procedure. The position of the maximum determines the echo shift parameter SPₓ for the respective subset of voxels. The same procedure is repeated for the determination of SP_{y} and SP_{z} in the remaining dimensions. The determination of the maxima separately for all three dimensions enables the composition of a vector representing the strength and direction of the (e.g. susceptibility induced) magnetic field gradient for the respective subset of voxels. The magnitudes of these vectors determined for all subsets of n voxels constitute the gradient map 22. The gradient map 22 has a n-fold reduced spatial resolution as compared to the MR image data set 21. By linear interpolation and by assigning grey values to the gradients 22, an image data set 24 with optimal positive contrast is generated. The image data set 24 can easily be adapted to weak and high susceptibility gradients via conventional image level and windowing operations. For visualization of the positive contrast induced by the magnetic field gradients, single slices of the data set 24 may be displayed by means of the display unit 15, as shown in FIG. 1. Alternatively, shim current values may be derived from the gradient map 22 and shim currents determined by the shim current values may be passed through shim coils 16, 17, 18 for producing an auxiliary magnetic field to optimze the homogeneity of the main magnetic field within the examination volume of the MR device 1.

1. A device for magnetic resonance imaging of a body placed in an examination volume, the device comprising
means for establishing a substantially homogeneous main magnetic field in the examination volume,
means for generating switched magnetic field gradients superimposed upon the main magnetic field,
means for radiating RF pulses towards the body,
control means for controlling the generation of the magnetic field gradients and the RF pulses,
means for receiving and sampling magnetic resonance signals, and
reconstruction means for forming MR images from the signal samples, the device being arranged to
a) generate a series of MR echo signals by subjecting at least a portion of the body to an MR imaging sequence of RF pulses and switched magnetic field gradients,
b) acquire the MR echo signals for reconstructing an MR image data set therefrom,
c) calculate a gradient map by computing echo shift parameters from subsets of the MR image data set, the echo shift parameters indicating local magnetic field gradient induced shifts of the echo positions in k-space, wherein each subset comprises a number of spatially adjacent pixel or voxel values of the MR image data set.

2. The device of claim 1, wherein the device is further arranged to
d) convert the gradient map into a positive contrast image by assigning grey values to the echo shift parameters.

3. The device of claim 1, wherein the device is further arranged to calculate the gradient map by computing Fourier transforms over the adjacent pixel or voxel values of each subset in step c).

4. The device of claim 3, wherein the device is further arranged to compute the echo shift parameters by determining the positions of the maxima of the Fourier components for each subset.

5. The device of claim 3, wherein the device is arranged to compute independent one-dimensional Fourier transforms over the adjacent pixel or voxel values in each spatial direction of the MR image data set.

6. The device of claim 5, wherein the device is arranged to calculate the gradient map by computing the strength and direction of the local magnetic field gradient from the echo shift parameters in the different spatial directions.

7. The device of claim 1, wherein the device is arranged to calculate the gradient map at a reduced spatial resolution as compared to the spatial resolution of the MR image data set.

8. The device of claim 1, wherein the device is further arranged to compute echo shift parameters from subsets comprising a small number of spatially adjacent pixel or voxel values of the MR image data set and to compute the echo shift parameters again from subsets comprising a larger number of spatially adjacent pixel or voxel values to improve accuracy.

9. The device of claim 1, further comprising shim coils for producing an auxiliary magnetic field to compensate for inhomogeneities of the main magnetic field, wherein the device is arranged to derive shim current values from the gradient map and to pass shim currents determined by the shim current values through each shim coil.

10. A method for MR imaging of at least a portion of a body placed in an examination volume of an MR device, the method comprising the following steps:
a) generating a series of MR echo signals by subjecting at least a portion of the body to an MR imaging sequence of RF pulses and switched magnetic field gradients,
b) acquiring the MR echo signals for reconstructing an MR image data set therefrom,
c) calculating a gradient map by computing echo shift parameters from subsets of the MR image data set, the echo shift parameters indicating local magnetic field gradient induced shifts of the echo positions in k-space, wherein each subset comprises a number of spatially adjacent pixel or voxel values of the MR image data set.

11. The method of claim 10, wherein the gradient map is converted into a positive contrast image by assigning grey values to the echo shift parameters.

12. The method of claim 10, wherein the gradient map is calculated by the following steps:
computing Fourier transforms over the adjacent pixel or voxel values of each subset in step c), and
computing the echo shift parameters by determining the positions of the maxima of the Fourier components for each subset.

13. The method of claim 10, wherein the gradient map is calculated at a reduced spatial resolution as compared to the spatial resolution of the MR image data set.

14. The method of claim 10, wherein shim current values are derived from the gradient map and shim currents determined by the shim current values are passed through shim coils for producing an auxiliary magnetic field to optimze the homogeneity of a main magnetic field within the examination volume.

15. A computer program for an MR device, comprising instructions for:
a) generating an MR imaging pulse sequence,
b) acquiring MR echo signals for reconstructing an MR image data set therefrom,
c) calculating a gradient map by computing echo shift parameters from subsets of the MR image data set, the echo shift parameters indicating local magnetic field gradient induced shifts of the echo positions in k-space, wherein each subset comprises a number of spatially adjacent pixel or voxel values of the MR image data set.

16. The computer program of claim 15, wherein the program further comprises instructions for converting the gradient map into a positive contrast image by assigning grey values to the echo shift parameters.

17. The computer program of claim 15, wherein the program further comprises instructions for deriving shim current values from the gradient map, which shim current values determine shim currents passed through shim coils of an MR apparatus.
